(19) United States
(12) Patent Application Publication
(10) Pub. No.: US 2008/0089839 A1
(43) Pub. Date: Apr. 17, 2008

(54) Preparation of Stable High Concentration Coloidal Metal Particulate System

(76) Inventors: Guo-Quan Lu, Blacksburg, VA (US); Guangyin Lei, Blacksburg, VA (US); Jesus Noel Calata, Blacksburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/573,303

(22) PCT Filed: Sep. 16, 2005
(86) PCT No.: PCT/US05/32620

§ 371 Date: Jan. 4, 2008

Related U.S. Application Data
(60) Provisional application No. 60/610,215, filed on Sep. 16, 2004.

(51) Int. Cl. A61K 49/00; C01B 33/14; A01N 59/16; A01N 59/20; A01P 1/00; A61K 47/02

(52) U.S. Cl. 424/9.1; 424/649; 424/618; 514/770; 514/769; 424/630; 424/650; 424/646

(57) ABSTRACT

Gold suspensions with high concentrations of gold, and silver suspensions with high concentrations of silver, are provided. Gold or silver nanoparticles are put into suspension, where the metal nanoparticles are paired with silica. Biocompatible suspensions are one application. Noble metal particles, attached to the surface of silica particles, form a stable, non-agglomerated suspension due to the steric and repulsive properties of the silica particles. The noble metal particles are prepared by activating the surface of the silica particles and erecting nucleation sites for metal particle growth, and then growing the metal particles at the nucleation sites through a reduction procedure.

### FIELD OF THE INVENTION

This invention relates to suspensions of metals, especially to noble metals such as gold or silver being present as particles in a suspension which may be used in a variety of applications.

### BACKGROUND OF THE INVENTION

Nano-structured metals have attracted widespread interest due to their size-dependent electronic and optical properties, which have led to numerous new applications including nano-electronics, photonic crystals, sensors based on surface enhanced Raman scattering (SERS) and near-field microscopy. Of the various metals, gold nanoparticles have already found wide application in the biomedical field because of their excellent biocompatibility and ease of bio-conjugation. On the other hand, silver nanoparticles are also broadly applied in detection and destruction of pathogens. Gold and silver nanoparticles with a diameter in the range of 10 to 30 nm exhibit distinctive absorption peaks in the 520-nm and 400-nm region of the optical spectrum, respectively. This is attributed to plasmon resonance at the metallic surface under optical excitation. This phenomenon is being exploited for labeling biological species.

Also, nanostructured metals are attracting widespread interest due to their significant size-dependent physical and chemical properties, which give rise to numerous applications including photonic crystals, sensors based on surface enhanced Ramon scattering and near-field microscopy. With fascinating optical and electrical properties, both gold and silver nanoparticles stand out as nearly perfect candidates for many of the applications. Another important property of gold nanoparticles that favors their use in many applications, especially in the biomedical fields, is their excellent biocompatibility and the ease of bio-conjugation. Colloidal gold particles, which are spherical and have a diameter of 15 nm, have a peak absorption wavelength at 520 nm. Besides the influences caused by the metal's dielectric property and the solvent's optical refraction index, the optical resonance is also a function of the size and the shape of the particles.

As one of the few materials with excellent biocompatibility and ease of bio-conjugation, gold nanoparticles are widely used in bio-detection, probing of DNA structures, drug and gene delivery, and biological labeling. On the other hand, in regard to anti-bacteria and detection of pathogens, silver nanoparticles are used due to their outstanding anti-microbial properties.

Conventionally, there have been certain preparations of pure gold or silver nanoparticles. Also, conventionally, there has been certain direct coating of silica nanoparticles by gold or silver. There has been much work on formation of metallic nanoparticles on large-sized silica particles, of over 150 nm in diameter, in an effort to coat the silica surface with a nanometer-thick layer of metal.

When suspended in de-ionized water, spherical gold and silver nanoparticles, with particle size smaller than 30-nm, possess an optical absorption spectrum peak at 520-nm and 400-nm, respectively. Besides the influences caused by the metal's dielectric property and the solvent's optical refraction index, the optical resonance is also a function of the size and the shape of the particles. Particles of different geometry, i.e. rods, rings or platelets, would have significant shifts of absorption spectrum away from the spherical particles. However, the sizes of these particles are much more difficult to control and thus the absorption spectrum seems to be much broader than the spherical particles. Therefore, most of the work being done is focused on spherical particles. Once the nanoparticles are synthesized, stabilization procedures should be carried out to overcome the particles' tendency to agglomerate. The most common way is performed by the surface modification processes, either by combining some stable polymers onto the particle surface or by coating the particle with some chemically inert inorganic materials, such as silica, to form core-shell geometry.

Thus, as noted above, a serious technical challenge facing the use of all nanoparticles has been attempting to prevent the particles from unwanted agglomeration. To try to achieve this, colloidal chemistry has been applied to prepare stable suspensions or colloids in which nanoparticles are kept apart by a layer of charged ions or organic molecules coated on the particles. Stable metal suspensions in water are readily available on the commercial market, but they are expensive and only available at low metal concentrations—too low to be useful for some applications.

In most cases conventionally, the stability of the high concentration gold colloidal, or silver colloidal, suspension is the key point to be solved. Although using some organic or inorganic chemicals may to some extent keep the colloidal suspension somewhat stable, conventionally workers have not succeeded in increasing the colloids' concentration much.

Prior efforts to coat the silica surface of nanometer thick layers of metal have included Journal of Colloid and Interface Sciences 263 (2003) 449-453; and Chem. Mater. 2001, 13, 1630-1633, which used ˜200-nm silica particles to have multiple deposits of metal particles up to about 10-nm in size. Their objective was to coat the silica particle surface with metal.

It should therefore now be appreciated that there exists an unmet need for a low-cost technique for making stable, high concentrations of metallic nanoparticles, especially of those metals receiving most focus for practical applications.

### SUMMARY OF THE INVENTION

An inventive technique has been now provided, in which a stable nanoscale silica suspension serves as a heterogeneous nucleation and stabilization medium. By controlling the processing conditions, the diameter of the metallic nanoparticles can be varied. Transmission electron microscopy (TEM) reveals the formation of metal-silica pairs stabilized in water over a wide range of pH. Ultraviolet (UV) and visible spectroscopy for products made according to the invention advantageously shows the distinctive absorption peaks in the optical spectrum, as found in gold and silver suspensions from commercial vendors of conventional products.

The invention provides a low cost method with good performance for preparing stable colloidal Gold-Silica and Silver-Silica suspensions (i.e., nanometal suspensions). A novel material-structure, nanosized Gold-Silica, or Silver-Silica, paired particles, has been invented.

In one preferred embodiment, the invention provides a method of making a metallic suspension, comprising the steps of: activating silica particles in a silica colloidal suspension so as to interact with noble metal ions (such as, e.g., silver, gold, platinum, and palladium); nucleating or otherwise attaching noble metal atoms (such as, e.g., silver, gold, platinum, palladium) to silica particles in said silica colloidal suspension; and growing noble metal particles on said silica particles at nucleation or attachment sites of said noble metal atoms (such as, e.g., a growing step that includes the step of adding a reducing agent to a solution containing activated silica particles and a metallic solution (such as, e.g., a metallic solution that comprises a buffer (such as, e.g., sodium bicarbonate)) containing one or more noble metals in salt form (such as, e.g., silver nitrate and hydrogen tetrachloroaurate)). In one embodiment, the step of activating is performed with tin chloride.

In another embodiment, the invention provides a metallic suspension, comprising: silica particles distributed within a carrier fluid; and noble metal particles (such as, e.g., one or more of silver, gold, platinum, and palladium) formed on surfaces of said silica particles, said noble metal particles having a size of 1-50 nm in diameter. Preferably, the inventive metallic suspensions further comprise a linker (such as, e.g., tin as an example) between said silica particles and said noble metal particles. Preferably in the inventive metallic suspensions the noble metal particles are present at a concentration ranging from about 10⁻³M to 10⁻²M. The inventive metallic suspensions preferably may be biocompatible, and may have biocompatible uses.

The invention also in another preferred embodiment provides a method of preventing biological contamination or destroying biological contaminants, comprising the steps of: providing silver particles (such as, e.g., silver particles that are distributed within a matrix (such as, e.g., an agar matrix); silver particles that are distributed as a suspension within a liquid carrier; etc.) in proximity to bacterial contaminants or a location where bacterial contamination may occur, said silver particles being associated with silica particles; and using the silver particles to prevent biological contamination or to destroy biological contaminants.

In another preferred embodiment, the invention provides a method of providing a biological marker to a patient, comprising the steps of: associating a gold-silica particulate material with a compound of interest; and administering said gold-silica particulate material with said compound of interest to a patient (e.g., injection, oral delivery, implanting, etc.), wherein gold in said gold-silica particulate material may be tracked as a marker after administration to said patient.

### BRIEF SUMMARY OF THE DRAWINGS

FIG. 1 is a flow chart showing preparation of a metal-silica suspension according to an embodiment of the invention.

FIGS. 2 and 2A are TEM images. FIG. 2 is for gold/silica paired particles, and FIG. 2A is for silver/silica paired particles, respectively, according to embodiments of the invention. The metallic nanoparticles in FIG. 2 are 20-nm size. FIG. 2A is for silver/silica paired particles, of 3-nm size.

FIGS. 2B and 2C are UV-visible absorption spectrum for gold/silica paired particles (FIG. 2B) and silver/silica paired particles (FIG. 2C), respectively, which are embodiments of the invention.

FIG. 3 is a transmission electron microscopic (TEM) image of exemplary inventive silver-silica coupling nanoparticles. The image magnification is 300,000. Particle size distribution histogram of silver nanoparticles is shown in the upper right corner.

FIG. 4 is a UV-vis absorption spectrum of exemplary inventive silver-silica composite nanoparticles. The spectrum is identical to pure silver nanoparticles, with absorption peak at 400-nm, and FWHM around 50-nm.

FIGS. 5A, 5B are bacterial growth curves showing Optical density versus time for Escherichia coli (FIG. 5A) and for Staphylococcus aureus (FIG. 5B), for an embodiment of the invention.

FIGS. 6A, 6B are exponential phase of bacterial growth curves, for Escherichia coli (FIG. 6A) and Staphylococcus aureus (FIG. 6B), for an embodiment of the invention.

FIGS. 7A, 7B are graphs for number of bacterial colonies as a function of the concentration of silver nanoparticles in the nutrient agar plates, in practicing an embodiment of the invention. FIG. 7A relates to Escherichia coli and FIG. 7B relates to Staphylococcus aureus. The photographs inserted show the agar plates containing different concentrations of silver nanoparticles. For Escherichia coli, they were (i) 0, (ii) 20, (iii) 30, and (iv) 60-mg/L. The data were (i) 0, (ii) 30, (iii) 60, and (iv) 120-mg/L for Staphylococcus aureus.

FIGS. 8A, 8B are graphs showing percentage of dead cells versus the concentration of certain inventive silver nanoparticles. FIG. 8A relates to Escherichia coli; dead cell percentages were 85±6%, 93±4%, and 97±2%, as the concentration of silver nanoparticles were 20-mg/L, 30-mg/L, and 60-mg/L, respectively. FIG. 8B relates to Staphylococcus aureus; dead cell percentages were 65±6%, 80±5%, and 91±3%, as the concentration of silver nanoparticles were 30-mg/L, 60-mg/L, and 120-mg/L, respectively.

### DETAILED DESCRIPTION OF A PREFERRED EMBODIMENT OF THE INVENTION

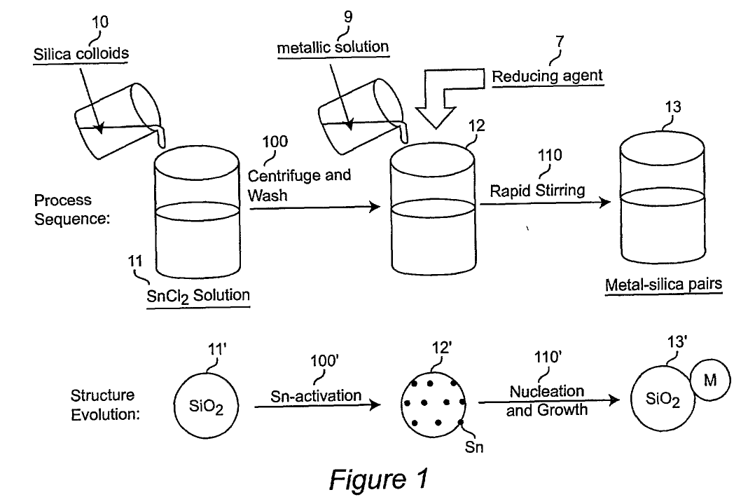

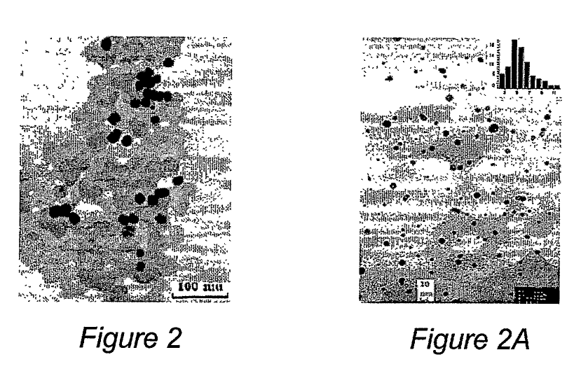

Referring to FIGS. 1-8B, preferred embodiments of the invention may be appreciated. First turning to FIG. 1, an inventive production process is shown, with the process sequence shown on the top line under which the corresponding structure evolution is shown on the bottom line. A starting material in the process sequence of FIG. 1 is silica colloids (or “particles”) 10. Silica particles can be obtained commercially, such as from catalog vendors (such as Alfa Aesar, Sigma-Aldrich, etc.)

Preferably the silica particles are 50-nm or less, such as a preferred range of about 14-nm to 20-nm average size.

It will be appreciated that silica particles are charged so as to repel one another in a suspension. Surface of silica particles may be modified (such as by sodium ions), however, from an overall particle charge point of view, these silica particles generally will remain negatively charged. In the invention, silica particles are used as nucleation sites and as physical barriers to prevent agglomeration or aggregation, and precipitation.

Silica colloids 10 are added to an activating solution, for example, a tin chloride solution 11. Another example of an activating solution may be a solution comprising a multi-valent metal salt with reduction potential. In the tin chloride solution, the silica has a structure of silicon dioxide 11′. The tin chloride solution then is centrifuged and washed 100. The process activates the surface of the silica, e.g., Sn-activation 100′, to yield silica particles 12′ that are activated with, e.g., tin at various points that can serve as nucleation sites for metal ions. Solution 12 includes structures 12′ which have undergone tin-activation 100′. In structure 12′ of FIG. 1, shows tin (Sn).

After centrifugation and washing 100, there is added thereto a metallic solution 9. For the metallic solution 9, a solution comprising noble metal ions is preferred, with a solution comprising gold or silver being most preferred (e.g., silver nitrate, hydrogen tetrachloroaurate, etc. may be present in metallic solution so as to provide silver and gold ions which will adhere to the silica particles 12′ at the activation sites). Some considerations in selecting a metal for the metallic solution 9 are as follows. Palladium, gold, or silver are typically used to activate non-conductive surfaces prior to electroless plating by a redox reaction with adsorbed tin ions. Copper can be deposited after activation with noble metals, such as in electroless copper deposition; however, in solution copper is easily oxidized.

The metallic solution 9 preferably includes a buffer, such as sodium bicarbonate, a weak base and salt combination (such as NH⁴⁺/NH³), etc.

To grow the metal ions adhered to the silica particles at the nucleation sites into metal particles (e.g., 01.-30 nm silver or gold particles), the solution 12 that includes the metallic solution 9 also receives reducing agent 7. The relative size of the metallic particles in relation to the size of the silica particles is not particularly limited, and particle size may be controlled by varying conditions. Examples of reducing agent are, e.g., formaldehyde, sodium borohydride, sodium citrate, etc. The choice of reducer depends on whether a strong (fast) or a slower reaction is wanted, which parameter may be used to control the particle size. Of the reducing agents formaldehyde, sodium borohydride, and sodium citrate, borohydride has the relatively strongest reducing ability, followed by formaldehyde, and citrate.

The particle size of the metal M adhered to the silica particle may be manipulated, such as, e.g., by switching among reducers; by rate of addition of reducers (with slower addition resulting in larger particles); and/or by the amount of metal salt initially present.

Solution 12 into which metallic solution 9 and reducing agent 7 have been added is subjected to rapid stirring 110 during which time the structural evolution is that of nucleation and growth 110′. The solution 12 is transformed into a suspension 13 comprising metal-silica pairs 13′. A metal-silica pair 13′ is composed of silicon dioxide and a metal particle M.

In suspension, such as a suspension 13, the silica particles continue to repel one another by virtue of their charge. From the overall particle charge point of view, the silica particles remain negatively charged (although the surface of the silica particles may have been modified, such as by sodium ions). The concentration of silica in the suspension 13 depends on the desired metal concentration, with a preferred example of a silica concentration range being, e.g., about 0.02 weight % to about 0.08 weight % in solution.

In suspension such as a suspension 13, a preferred example of a concentration range for the metal in the suspension is about 1*10⁻³ to about 1*10⁻²M.

A shape of the particles which are formed on the silica surfaces is generally spherical as this is the most energetically stable geometry. However, shapes have been observed which are distorted spheres.

Stable suspensions may be formed with particle sizes up to about 30-nm, with a preferred size for the particles formed in a range of about 1-nm to 5-nm.

Examples of applications for the inventive metal-silica suspensions and solutions include, for example, Precise Controlled Drug Delivery and Drug Targeting; Real-Time Optical Biosensor; Optical switching; Optical filters; Biological sensors; Directly used as nutritional supplement to improve human's mental performance; Directly used as drug in the Treatment of Rheumatoid Arthritis (RA); Bio-detection of pathogens; etc.

In inventive suspensions comprising metal particles (such as metal nanoparticles) paired with silica (such as silica nanoparticles), the respective contents of metal and silica may be adjusted to provide multi-functioning applications.

Using the inventive productions processes, such as a process according to FIG. 1, an inventive colloidal metal suspension (such as, e.g., a gold or silver suspension) may be prepared that has a high metal concentration, such as a metal concentration that exceeds about 5×10⁻³M.

The invention may be further appreciated with respect to the following Examples, without the invention being limited thereto.

### EXAMPLE 1

In order to form the paired particles, tin (II) chloride (SnCl₂) was used as a linker between silica and metallic particles. Initially, when mixing the silica colloids and SnCl₂ solution together, Sn²⁺ ions activate the silica surface by replacing the OH⁻ groups. Afterwards, aqueous solution containing hydrogen tetrachloroaurate (HAuCl₄.2H₂O) or silver nitrate (AgNO₃), and sodium carbonate (Na₂CO₃) was added into the Sn-activated silica suspension. As the Sn²⁺ ions are oxidized by metallic ions, either Au³⁺ or Ag⁺, some metallic atoms attach onto the silica surface, and this can be referred to as the nucleation process. Later on, proper reducers, which would further reduce the metallic ions to metallic atoms, were added into the solution. With more and more metallic atoms reduced out, they would aggregate together to form bigger particles on the surface of silica particles, resulting in metal-silica paired particles of the invention.

The prepared gold-silica, or silver-silica paired particles were found to be stable under a wide range of pH for several months. The metallic nanoparticle size could be easily controlled by adjusting the ratio of the amount of metallic ions and the number of silica particles, in combination of choosing the proper reducing agents. For small metallic nanoparticles, less than 10-nm, sodium borohydride (NaBH₄) was found effective to maintain the uniformity in particle size. However, for larger particles, fresh formaldehyde should be applied. Its weak reducing ability will slow down the redox reaction, and thus extend the particles' growing phase.

By successfully employing the colloidal silica particles as carriers of metallic nanoparticles, the invention provides an easy way to prepare high concentration colloidal suspensions. The suspensions are quite stable in a relatively broad pH range with narrower particle size distribution compared to the existing technology.

As is well known, silica particles are chemically inert and optically transparent, such that it will not affect the optical properties of the metallic nanoparticles. Moreover, high concentration colloidal silica suspension can be easily prepared by using sodium oxide for surface modification and is also commercially available. Numerous applications have already used silica particles in tailoring the optical properties of some metallic nanoparticles. Through our method, colloidal gold, or silver particles can be effectively attached to the silica particles' surfaces, and thus form the metal-silica pairs. The prepared suspension makes use of the strong repulsive force among the commercial colloidal silica particles, as well as using the silica as steric barrier, which greatly enhances the colloidal system's stability even with high concentration.

### EXAMPLE 2

In this Example, silver nanoparticles were synthesized in an aqueous suspension of silica nanoparticles. With silver nanoparticles anchored on silica surface, suspensions were found to be stable at high silver concentrations as well as over a broad pH range. The antimicrobial activities of these composite nanoparticles were investigated. Escherichia coli and Staphylococcus aureus were used as representatives of Gram-negative and Gram-positive bacteria respectively. Bacteriological tests data showed either bacterial growth inhibition or cell death occurred, corresponding to different concentrations of silver nanoparticles. Transmission electron microscopy (TEM) was used to reveal the morphology and the size of the silver-silica coupling nanoparticles. Fluorescent microscopic images were provided to confirm the bacterial viability after three hours' treatment with silver nanoparticles.

Nanostructured materials have been the focus of intense research in past decades due to the significant size-dependent changes in their physical and chemical properties. The size of such particle can be tailored from 0.1 nm to 100 nm in diameter with moderate to excellent control over size dispersity, depending upon chosen composition. The novel properties of these nanoparticles can be taken advantage of for optical and electrical applications, including nano-electronics, photonic crystals, sensors based on surface enhanced Raman scattering (SERS) and near-field microscopy. However, knowledge to date in biological and anti-pathogen properties of these nanostructured materials is still limited, especially in the anti-bacteria field. Some of the recent literature reported the encouraging results of bactericidal properties of several nanostructured materials. Hamouda et al. revealed the broad-spectrum sporicidal activity of certain nanoemulsions, which were also found to be stable, easily dispersed, nonirritant, and nontoxic compared with other agents. Klabunde et al. reported that when significantly adsorbed with halogen (C1₂, Br₂), Magnesium oxide (MgO) was very effective against Gram-positive and Gram-negative bacterial cells as well as spores.

As is well known, some elements by themselves are malignant to microorganisms, and silver is the most toxic. Therefore, silver ions and silver containing products are widely used in medical applications. For instance, silver compounds are used for treatment of serious burns; in bandages for trauma and diabetic wounds; and are used to coat the catheters and medical devices to prevent the growth of bacterial biofilms. Numerous researches have done to reveal the mechanisms of bactericidal property and even bacterial resistance of silver ions. Not until recently, Sondi et al. reported the antimicrobial property of silver nanoparticles against E. coli, which is a gram-negative bacterium; it was, to our knowledge, the first literature on the study of bactericidal property of nanostructured metal. However, from the efficiency point of view, their experiments were limited due to the non-ideal stability of the silver nanoparticles. In this regard, a low-cost technique is provided for making stable aqueous silver suspension with high concentration of silver nanoparticles, extensive bactericidal tests against E. coli and Staphylococcus aureus, and confirmed the bactericidal property of silver nanoparticles.

With these considerations, the invention in this Example provides a technique that uses a stable nanoscale silica suspension to serve as a heterogeneous nucleation and stabilization medium. By varying the processing conditions, diameter of the silver particles can be controlled. Transmission electron microscopy (TEM) reveals the formation of silver-anchored silica nanoparticles, which stabilized in water over a broad range of pH. Ultraviolet and visible spectroscopy (UV-vis) shows the distinctive absorption peaks in the optical spectrum. As silica nanoparticles are chemically inert and biologically benign, they are not supposed effect the bactericidal tests, which has also been confirmed from our experimental data. Silver-anchored silica nanoparticles have been synthesized by the surface modification method via tin sensitization and silver activation of the silica nanoparticles. The silver particles are directly adsorbed onto the silica surface by the reduction and deposition processes, with a controllable diameter from 2-nm to 20-nm (and may be operable at other dimensions e.g. 0.1-50 nm). This nanostructure takes advantage of silica nanoparticles as steric barrier as well as the strong electrical repulsive force that silica particles posses, thus the resulting colloidal suspension has greatly enhanced stability.

### Experimental

### Materials and Chemicals

Silver nitrate (AgNO₃, ACS, 99.9% metal basis) and colloidal silica suspension (14-nm, 40-wt % in water) were supplied by Alfa Aesar. Sodium borohydride (NaBH₄) and tin (II) chloride (SnCl₂) were obtained from Sigma Aldrich. All chemicals were used as received. LIVE/DEAD BacLight Bacterial Viability Kits L7007 was obtained from Molecular Probes, and stored in −40° C. before the fluorescence microscopic observation. All glassware used in the synthesis of silver nanoparticles were cleaned with aqua regia (3 parts HCl, 1 part HNO₃), rinsed with 18.3-MΩ nano-pure water, and dried in oven prior to use.

### Cultures

Escherichia coli strain B and Staphylococcus aureus were obtained from Presque Isle Cultures, PA. The components of the Luria-Bertani (LB) medium, Tryptic Soy Broth, and agar solidifying powder were purchased from Difco Laboratories.

Preparation of Silver Nanoparticles

Colloidal silica suspension contains sodium oxide to keep the colloidal system stable and has a solid loading of 40 percent by weight of silica. A stock solution was prepared by adding9-ml of the silica colloids to 150-ml of 2.5×10⁻³M tin (II) chloride (SnCl₂) solution and stirred for half an hour for the initial activation of silica surface. The reaction believed to occur is as follows:

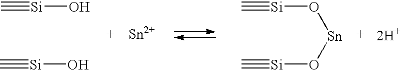

The Sn-activated silica particles were then centrifuged and washed with deionized water three times to get rid of the excess Sn²⁺ and other residuals. 1-ml of this Sn-activated silica solution was further diluted to 50-ml with deionized water. Afterwards, 20-ml of silver nitrite (AgNO₃, 0.005-M) was freshly prepared, and mixed with the aforementioned 50-ml of silica suspension under rapid stirring for about 30-minutes. On the other hand, fresh sodium borohydride (NaBH₄) was prepared by dissolving 0.4-g of NaBH₄ powder into 25-ml of deionized water, and 2-ml of which were then added into the silver-silica mixture dropwisly during a time period of 10 minutes, which would ensure the depletion of silver ions. Upon the addition of the first drop of sodium borohydride, the color of the suspension changed dramatically from pale yellow to dark yellow, even showed some brownish. It was observed that as more sodium borohydride were added afterwards, more and more silver ions were reduced, and the color became less intense. The suspension later even turned to slightly clear, as resulting from the decrease of the number of silver nanoparticles, which due to the consuming of small nucleus in forming the larger particles. The samples were then examined by transmission electron microscopy (TEM) to determine the particle size and morphology of the reduced silver while UV-visible spectroscopy was used to obtain the optical spectrum data.

### Bacteriological Tests

Bacterial Growth Tests

To examine the overall effects of silver nanoparticles on bacteria, liquid medium tests were conducted as follows. About 10-ml suspensions of either Escherichia coli in LB or Staphylococcus aureus in Tryptic Soy Broth were cultured overnight, to late log phase in nutrient broth. Their optical densities at 600-nm were determined via the bench top “Genesys 10” UV-vis spectrophotometer, using a 1 ml aliquot of the bacterial suspension in an acrylic cuvette. Based on calculation, certain volume of cells was then transferred to 50-ml of nutrient medium, to make the starting optical density as 0.05, which corresponds to about 2.5*10⁶ cells/ml. The nutrient mediums were afterwards mixed with silver suspensions, and the concentrations of silver nanoparticles were arbitrarily chosen range from 0-mg/L to 120-mg/L. Since silver nanoparticles also absorb light at 600-nm, 1 ml of each silver containing suspension, were kept to determine the baseline of optical density. The cultures were then incubated at 37° C. for up to 10 hours. The optical density (OD) data were taken every 25 minutes for Escherichia coli and 40 minutes for Staphylococcus aureus.

Bacterial Viability Tests

Bacterial viability was investigated in two approaches, one of which was to conduct on nutrient agar plate. By counting the bacterial colonies live cells formed using the cultures treated with silver nanoparticles, those bactericidal effects could be concluded briefly. Another approach was based on the fluorescent microscopy observation, which distinguished live and dead cells to generate bacterial death rates.

### Nutrient Agar Plate Tests

Ten milliliters of either Escherichia coli or Staphylococcus aureus were cultured, following the same procedure as described previously. Then the bacterial cells were concentrated by centrifugation at 10,000-g for 15 minutes. Supernatant was removed and the pellet was resuspended in 10 ml of PBS buffer solution, and incubated at room temperature for 1 hour, mixing every 15 minutes. Repeat the centrifugation and resuspension steps twice, before the optical density was adjusted to 1.0. By applying this protocol, live bacteria could be separated from the nutrients, thus to keep the total cell number constant since the cells could not duplicate. Subsequently, colloidal suspensions with different concentrations of silver nanoparticles were mixed with the bacteria, and cultured at 37° C. for 3 hours. The suspensions were then diluted with PBS buffer solution, to generate 250-ul of samples, which were placed on the surface of the nutrient agar. These agar plates were then incubated at 37° C. overnight. Based on counting the colonies that formed later, the number of killed bacteria during this time period of 3 hours could be derived, as the effects of silver nanoparticles were considered greatly limited when the suspensions were placed on the surface of the agar.

### Fluorescence Microscopy Experiments

Fluorescence microscopy was carried out as an alternative to determine the bacterial viability. The bacterial cells were cultured similarly as described previously. After being cultured for three hours, cell suspensions were firstly being centrifuged to remove the silver nanoparticles. Suspensions were then mixed with the LIVE/DEAD bacterial viability molecular dyes, and stored in dark for 15 minutes. Live or dead cells can be differentiated by the integrity of cell membranes, which can be manifested through dye binding—green for intact cell membrane and red for damaged membrane. Therefore, the fluorescence microscopic images could be used as direct proofs to differentiate the dead or live cells. Texas Red bandpass filter sets were applied in our experiment, for tuning the excitation fluorescence wavelengths. Briefly, 6-ul of stained bacterial suspension was trapped between a slide and a 24-mm square coverslip. Under the help of mounting oil, optimal magnitude could be chosen. During the course of observation, filters could be changed due to different observation purposes.

### Ultraviolet and Visible Spectroscopy (UV-VIS) Experiments

Optical absorption spectrum of the silver-silica coupling suspensions was obtained by a Shimadzu UV-3101 PC UV-vis-NIR scanning spectrometer in the wavelength range from 400-nm to 700-nm. And “Genesys 10” UV-vis spectrophotometer was used in the measurements of the optical density of the bacterial cultures in liquid nutrient medium, with the measuring wavelength set at 600-nm. Quartz cuvettes with optical path length of 10 mm were used in both measurements.

### Transmission Electron Microscopy Experiments

Philips 201 transmission electron microscopy was used for the characterization of silver-silica coupling nanoparticles, and was operated at 80-kV accelerating potential. Small amount of colloidal samples were deposited on bare 200 mesh copper grids, and dried in air with cover prior for observation.

### Results

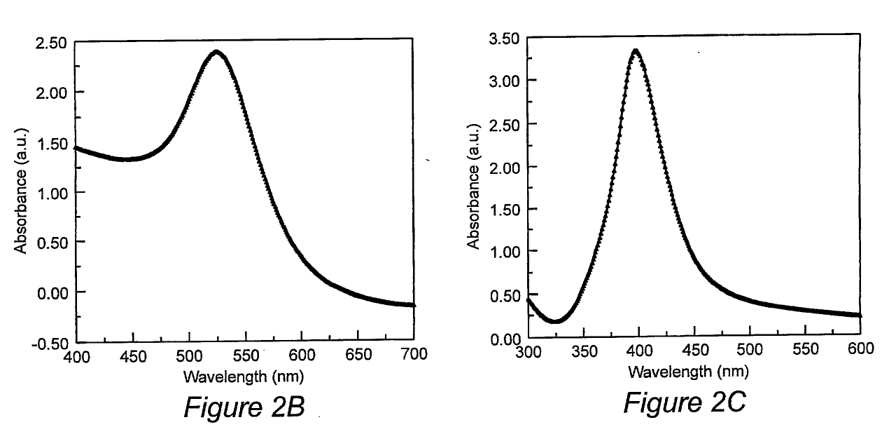

Highly concentrated silver-silica coupling suspension was synthesized by applying the above Morphology and size of the silver nanoparticles were characterized by transmission electron microscopy; the image was shown in FIG. 3. Due to the higher optical density compared with silica nanoparticles, the silver nanoparticles were found much darker than the silica nanoparticles, which could be identified in the TEM image. Most of the silver nanoparticles were spherical, attached to the silica surfaces, yet some detached particles could also be found. In regard of the particle size, silver nanoparticles were varying between ˜2-nm and ˜6-nm, with a mean diameter around 5-nm. Corresponding particle size distribution histogram of the obtained nanoparticles was analyzed by Image-pro plus, and was given in the upper right corner of FIG. 3.

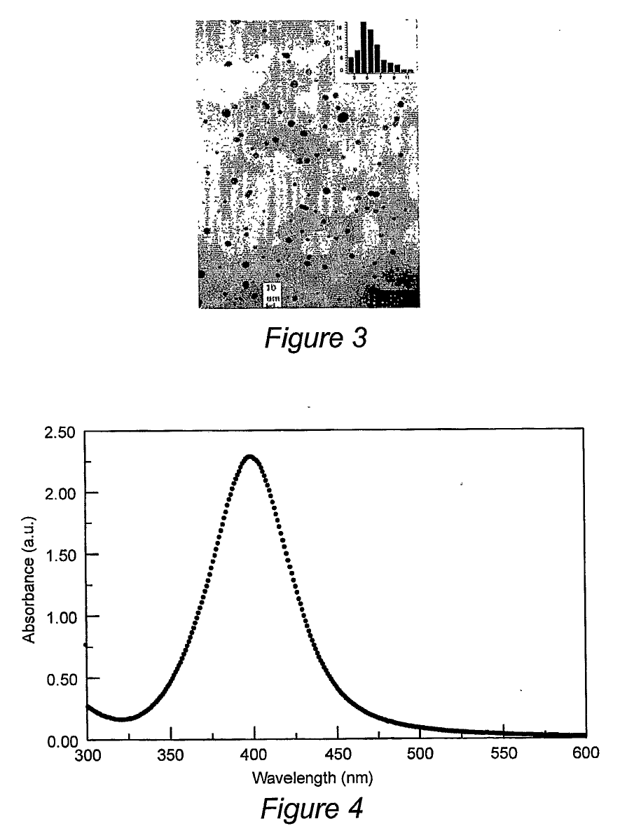

Another characteristic of the silver nanoparticles, the UV-vis absorption spectrum, is illustrated in FIG. 4. The optical spectrum showed a well-defined plasmon band at 400-nm, with full width at half maximum (FWHM) of around 50-nm, which was identical to the pure silver colloids. And this also confirmed our postulation that although some light scattering might occur due to the presence of silica nanoparticles, the absorption spectrum of the silver nanoparticles, especially the absorption peak, would not be influenced much.

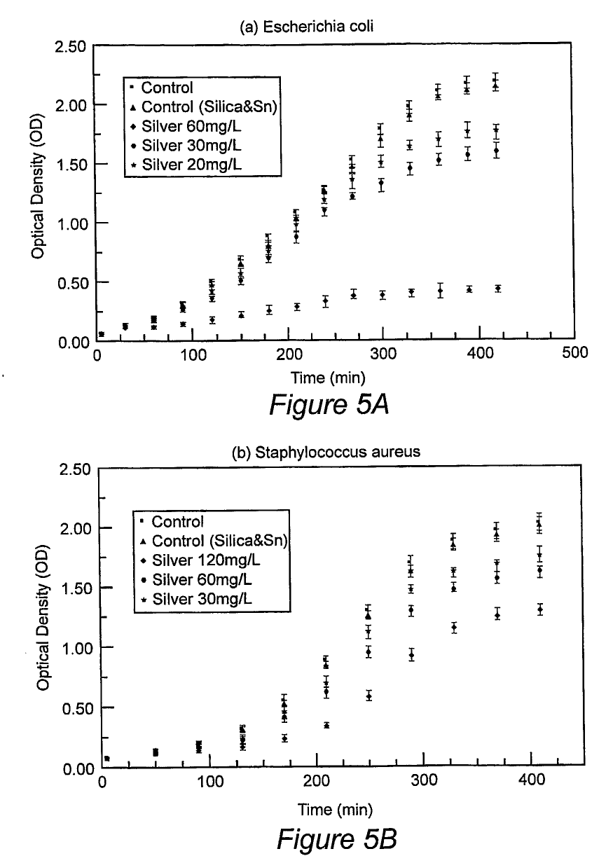

Bactericidal tests were conducted against two strains as representatives of different bacterial types. Escherichia coli were used as the representative of Gram-negative bacteria, and Staphylococcus aureusfor Gram-positive bacteria. As described in the former section, bacterial growth tests were performed to study the overall bactericidal effects of the silver nanoparticles. The concentrations of silver nanoparticles were chosen as 60-mg/L, 30-mg/L, and 20-mg/L for the tests against Escherichia coli; and 120-mg/L, 60-mg/L, and 30-mg/L for the test against Staphylococcus aureus, respectively. Bacterial OD data were collected for the determination of bacterial population number (FIG. 5).

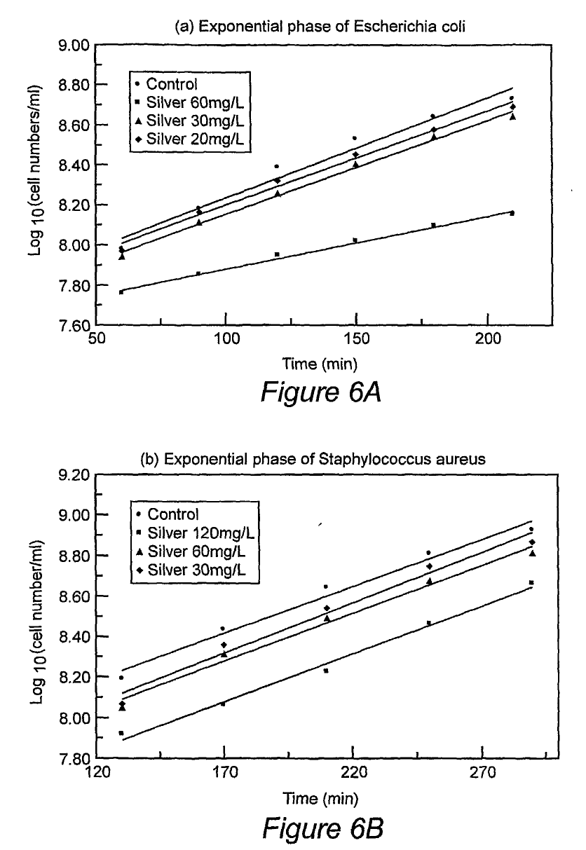

The bacterial growth curves showed significant difference crossing the whole four phases on both Escherichia coli and Staphylococcus aureus. To exclude the potential bactericidal effects brought by the auxiliary chemicals in the colloidal silver suspension, an extra control sample was prepared by mixing all the chemicals, except silver component. It was obvious that this extra control sample had an almost identical growth curve with the real control sample, which contained the nutrient medium only. Therefore, we concluded that the bactericidal effects of the colloidal mixture were only brought by the silver nanoparticles. However, from the bacterial cell number and growth rate viewpoints, transformation of the linear ordinate to the exponential ordinate was applied. Several consecutive points were afterwards picked as the presentations of the exponential phase (FIG. 6).

Differences between the control sample and the samples with silver nanoparticles were also significant. In addition, one notable discrepancy between two tests, between that against Escherichia coli and that against Staphylococcus aureus, was the bacterial growth rate, especially under high concentrations of silver nanoparticles. In the test against Escherichia coli, when the concentration of silver nanoparticles was raised up to 60-mg/L, the slope of the bacterial growth curve was almost only half of the number derived from the control sample. Therefore in terms of bacterial generation time, that would be two times longer than the normal Escherichia coli. On the other hand, this growth inhibition did not show in the test against Staphylococcus aureus, in which the bacterial growth curves were almost parallel to each other, regardless of the concentration of silver nanoparticles. We concluded this discrepancy was probably due to the significant differences in the cell wall structure of these two bacteria.

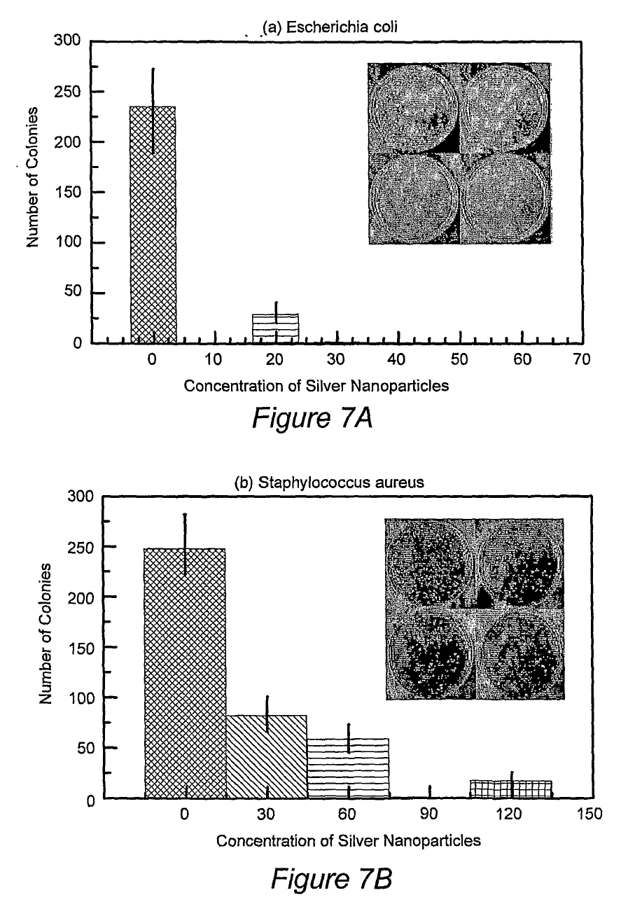

Bacterial viability tests were conducted in order to determine the proportions of viable bacteria after treatment with silver nanoparticles, in a quantitative approach. For the nutrient agar tests, as described in the previous section, bacteria were cultured under different concentrations of silver nanoparticles, and afterwards about 300 bacterial cells were placed on the agar plates. The numbers of bacterial colonies were counted 12 hours later, as shown in FIG. 7.

Meanwhile, for the test against Escherichia coli, we found that the numbers of colonies reduced significantly in the presence of silver nanoparticles. About 100%, 98±2%, and 88±3% of bacteria were killed, when the concentrations of silver nanoparticles were 60-mg/L, 30-mg/L, and 20-mg/L respectively. In addition, tests were also conducted with increased the number of bacterial cells, placed on the agar surface, by the order of two magnitudes. While the numbers of colonies were too many to count in the other agar plates, there still no colony formed in the sample with 60-mg/L silver nanoparticles. The nutrient agar plate tests against Staphylococcus aureus generated different results. Small proportions of colonies were still formed even in the sample with the highest concentration of silver nanoparticles, 120-mg/L. Only 93±3%, 77±4%, and 67±4% cells were killed after the treatment, with respect to the silver concentrations of 120-mg/L, 60-mg/L, and 30-mg/L.

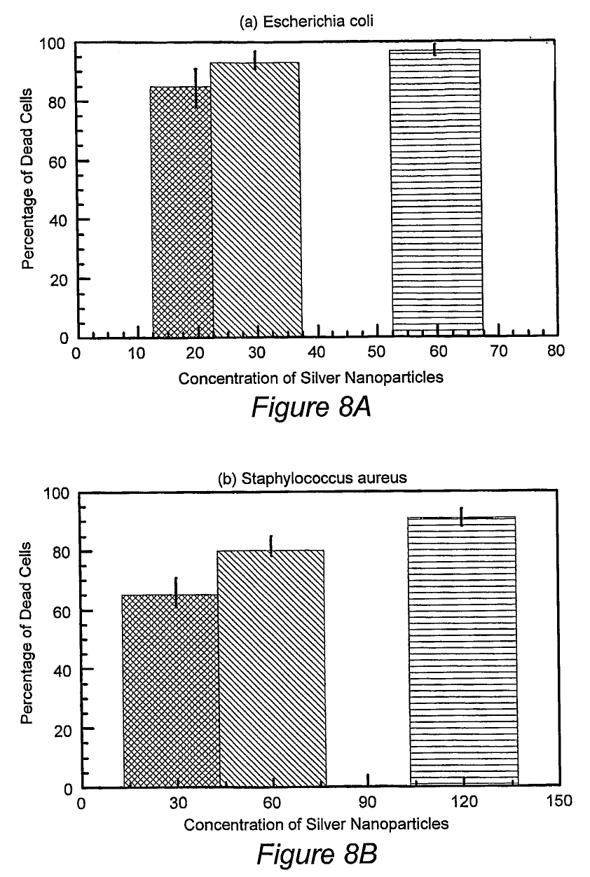

By laser fluorescence microscopy, the bacterial viable proportions were determined. For each sample, two images were taken at exactly the same image spot, with different fluorescence excitation wavelength and corresponding bandpass filters, so dead cells could therefore be recognized from the overall population by comparing the two images. However, since the images that we captured from the optical camera only showed black and white, they were later tainted with red and green by using “Image-pro Plus”. As a consequence, after we overlapped the images together, the dead cells would to some extent appear as orange or even yellow, depended upon the light intensities of the images. Cell numbers were also counted by using “Image-pro plus” (see FIG. 8). As the percentages of dead cells were calculated and compared with the numbers derived from the nutrient agar plate tests, only small differentials were noticed.

In summary, the antimicrobial properties of silver nanoparticles were investigated against Escherichia coli and Staphylococcus aureus, which represent Gram-negative and Gram-positive bacteria respectively. Large proportions of bacteria were killed upon treatment with silver nanoparticles. In addition, with even low concentrations of silver nanoparticles, bacteria growth inhibition could occur, which also greatly reduced the amount of live bacteria comparing with the control sample.

While the invention has been described in terms of its preferred embodiments, those skilled in the art will recognize that the invention can be practiced with modification within the spirit and scope of the appended claims.

What we claim as our invention is:

1. A method of making a metallic suspension, comprising the steps of:
activating silica particles in a silica colloidal suspension so as to interact with noble metal ions;
nucleating or otherwise attaching noble metal atoms to silica particles in said silica colloidal suspension; and
growing noble metal particles on said silica particles at nucleation or attachment sites of said noble metal atoms.

2. The method of claim 1 wherein said growing step includes the step of
adding a reducing agent to a solution containing activated silica particles and a metallic solution containing one or more noble metals in salt form.

3. The method of claim 2 wherein said one or more noble metals in salt form are selected from the group consisting of silver nitrate and hydrogen tetrachloroaurate.

4. The method of claim 2 wherein said metallic solution comprises a buffer.

5. The method of claim 4 wherein said buffer is sodium bicarbonate.

6. The method of claim 1 wherein said noble metal particles are selected from the group consisting of copper, silver, gold, platinum, palladium, and iridium.

7. The method of claim 1 wherein said step of activating is performed with tin chloride.

8. A metallic suspension, comprising:
silica particles distributed within a carrier fluid; and
noble metal particles formed on surfaces of said silica particles, said noble metal particles having a size of 1-50 nm in diameter.

9. The metallic suspension of claim 8 further comprising a linker between said silica particles and said noble metal particles.

10. The metallic suspension of claim 9 wherein said linker is tin.

11. The metallic suspension of claim 8 wherein said noble metal particles include one or more of copper, silver, gold, platinum, palladium, and iridium.

12. The metallic suspension of claim 8 wherein said noble metal particles are present at a concentration ranging from 10⁻³M to 10⁻²M.

13. The metallic suspension of claim 8 wherein the suspension is biocompatible.

14. A method of preventing biological contamination or destroying biological contaminants, comprising the steps of:
providing silver particles in proximity to bacterial contaminants or a location where bacterial contamination may occur, said silver particles being associated with silica particles; and
using the silver particles to prevent biological contamination or to destroy biological contaminants.

15. The method of claim 14 wherein said silver particles are distributed within a matrix.

16. The method of claim 15 wherein said matrix is agar.

17. The method of claim 14 wherein said silver particles are distributed as a suspension within a liquid carrier.

18. A method of providing a biological marker to a patient, comprising the steps of:
associating a gold-silica particulate material with a compound of interest; and
administering said gold-silica particulate material with said compound of interest to a patient, wherein gold in said gold-silica particulate material may be tracked as a marker after administration to said patient.
